(19) United States
(12) Patent Application Publication
(10) Pub. No.: US 2024/0393312 A1
(43) Pub. Date: Nov. 28, 2024

(54) METHOD FOR DETERMINING ODOR DETERIORATION AND LIPID OXIDATION DEGREE OF TILAPIA DURING COLD STORAGE

(71) Applicant: Shanghai Ocean University, Shanghai (CN)

(72) Inventors: Jing XIE, Shanghai (CN); Hao CHENG, Shanghai (CN); Jinfeng WANG, Shanghai (CN); Jun MEI, Shanghai (CN)

(21) Appl. No.: 18/795,923

(22) Filed: Aug. 6, 2024

(30) Foreign Application Priority Data
May 25, 2024 (CN) 202410661115.9

(51) Int. Cl. G01N 33/12; G01N 1/40; G01N 1/42; G01N 30/72

(52) U.S. Cl. CPC G01N 33/12; G01N 1/4077; G01N 1/42; G01N 30/7233; G01N 2001/4088

(57) ABSTRACT

A method for determining odor deterioration and lipid oxidation degree of tilapia during cold storage is provided. Fresh tilapia fillets are collected, cleaned, mixed and minced. A lipid extract is obtained, and divided into multiple lipid samples. The lipid samples are refrigerated for different days and analyzed by a lipidomics technique to obtain lipid oxidation degree and lipid metabolism data. A lipid metabolic network associated with odor is established. The lipid metabolic data is analyzed based on the lipid metabolic network to obtain metabolic markers corresponding to the lipid samples. An evaluation metabolite marker is screened among the metabolic markers. The metabolic status of the lipid samples is analyzed based on the evaluation metabolite marker.

### CROSS-REFERENCE TO RELATED APPLICATIONS

This application claims the benefit of priority from Chinese Patent Application No. 202410661115.9, filed on May 25, 2024. The content of the aforementioned application, including any intervening amendments made thereto, is incorporated herein by reference in its entirety.

### TECHNICAL FIELD

This application relates to fish odor research, and more particularly to a method for determining odor deterioration and lipid oxidation degree of tilapia during cold storage.

### BACKGROUND

Tilapia is a popular edible fish that has rapid growth and reproduction, delicious taste and abundant nutrients. Due to the high water content, fresh tilapia is prone to deterioration under the action of microorganisms and enzymes. Therefore, the fresh tilapia is normally stored at a low temperature. However, with the extension of refrigeration time, the product quality will gradually deteriorate, accompanied by the generation of undesirable odor, which seriously affects the production, processing and application of tilapia. In view of this, the investigation of the odor of tilapia during the cold storage has attracted considerable attention. It has been found that the odor change is mainly related to volatile compounds, which can be produced in many biochemical processes, such as lipid oxidation, protein degradation and microbial action. In particular, the lipid oxidation has been widely considered as a key pathway for the formation of characteristic odors. Lipid oxidation is a complex process involving the oxidation of free fatty acids (FFAs) and the action of free radicals. As a class of important flavor precursors, FFAs also contribute to the flavor diversity. However, the correlation between the FFAs and odors has still not been reported, which limits the determination of fish quality and odor deterioration.

### SUMMARY

An object of the disclosure is to provide a method for determining odor deterioration and lipid oxidation degree of tilapia during cold storage, so as to solve the technical problems existing in the prior art.

In order to achieve the above object, the following technical solutions are adopted.

This application provides a method for determining odor deterioration and lipid oxidation degree of tilapia during cold storage, comprising:
step (1) killing fresh tilapia followed by cutting to obtain a plurality of tilapia fillets, washing the plurality of tilapia fillets followed by wiping to remove surface water, and subjecting the plurality of tilapia fillets to mixing and mincing to obtain a raw material;
step (2) extracting a lipid extract from the raw material;
step (3) dividing the lipid extract into a plurality of lipid samples, and refrigerating the plurality of lipid samples respectively for different times; and after refrigeration, determining lipid oxidation degree and lipid metabolism data of each of the plurality of lipid samples by using a liquid chromatography-mass spectrometry (LC-MS)-based lipidomics technique;
step (4) establishing a lipid metabolic network associated with odor through a combination of a kyoto encyclopedia of genes and genomes (KEGG) pathway database and a metabolomics pathway analysis (MetPA) database, and analyzing the lipid metabolic data of each of the plurality of lipid samples based on the lipid metabolic network, so as to obtain metabolic markers corresponding to the plurality of lipid samples; wherein the lipid metabolic data comprises a metabolite and a metabolic pathway;
step (5) screening an evaluation metabolite marker among the metabolic markers based on the following criteria: p-value ≤0.05 and variable importance in projection (VIP)≥1; and
step (6) analyzing a metabolic status of each of the plurality of lipid samples based on the evaluation metabolite marker.

In some embodiments, the step (1) is performed through a step of:
placing the fresh tilapia in a foam box with crushed ice and oxygen for transportation, stunning the fresh tilapia by physical knocking, killing the fresh tilapia followed by removal of head and internal organs and cutting to obtain the plurality of tilapia fillets, washing the plurality of tilapia fillets followed by wiping to remove surface water; and mixing and mincing the plurality of tilapia fillets to obtain the raw material.

In some embodiments, the step (2) is performed through steps of:
step (2.1) placing 5 g of the raw material in a 50 mL centrifuge tube followed by addition of 15 mL of a chloroform-methanol mixture containing 0.01 wt. % (0.01 g/100 g) of butylated hydroxytoluene to obtain a first mixture; wherein a volume ratio of chloroform to methanol is 2:1;
step (2.2) sealing the 50 mL centrifuge tube with a stopper, and grinding the first mixture twice at 10,000×g in an ice bath for 10 s, 13 s, 15 s or 20 s;
step (2.3) diluting the first mixture to 30 mL, followed by standing for 45 min, 55 min, 65 min or 75 min and filtration to obtain a filtrate; and
step (2.4) adding a saline to the filtrate to obtain a second mixture, wherein a volume ratio of the saline to the filtrate is 0.2:1, and a concentration of the saline is 0.85 g/100 g; centrifuging the second mixture at 3,000×g for 10 min, 12 min, 15 min, 17 min or 20 min for layering; and collecting a bottom-layer phase followed by drying in a nitrogen flow to obtain the lipid extract.

In some embodiments, in step (3), the lipid extract is divided into four lipid samples, and the four lipid samples are refrigerated at 4° C. for 0 day, 3 days, 9 days and 15 days, respectively.

In some embodiments, in step (3), the LC-MS-based lipidomics technique is performed through steps of:
adding 0.5 mL of each of the plurality of lipid samples to a 2 mL centrifuge tube followed by addition of 600 μL of a methanol solution containing 4 ppm of 4-chloro-L-phenylalanine maintained at −20° C. and vortex mixing for 30 s to obtain a mixture;
homogenizing, by a tissue homogenizer containing 100 mg of glass beads, the mixture at 60 Hz for 90 s;
centrifuging the mixture at 12,000×g and 4° C. for 10 min followed by ultrasonic treatment at room temperature for 7 min, 8 min, 10 min, 12 min or 13 min, and collecting a supernatant; and filtering the supernatant with a 0.22-μm membrane filter to obtain a filtrate; and
transferring the filtrate to a vial followed by LC-MS analysis and chromatography.

In some embodiments, the step (6) is performed through a step of:
randomly selecting three lipid samples among the plurality of lipid samples respectively on day 0, day 3, day 9 and day 15 as parallel groups to analyze lipid oxidation degree and lipid metabolism.

In some embodiments, the evaluation metabolic marker is selected from the group consisting of 1-hexadecanol, 1-monopalmitate, 10-heptadecenoic acid, 13-docosenamide, 5,8,11-eicosatrienoic acid, 7,10,13,16,19-docosapentaenoic acid, 9-octadecenoic acid, 9,12-octadecadienoic acid, arachidonic acid, ethyl 4-ethoxybenzoate, campesterol, dibutyl phthalate, glycerol monostearate, heptadecanoic acid, myristic acid, oleic acid, palmitelaidic acid, palmitic acid and a combination thereof.

Compared to the prior art, the present disclosure has the following beneficial effects.

In the present disclosure, the lipid extract is extracted, and stored in a refrigerated environment for a certain number of days. A lipid metabolic network associated with the odor of tilapia is established through the analysis of the LC-MS-based lipidomics technique. The evaluation metabolite marker is screened, and lipid oxidation degree and the deterioration of the odor quality are determined through a content of the evaluation metabolite marker.

In the present disclosure, the lipid metabolic network diagram associated with odor is constructed, and an association between lipid metabolism and odor deterioration is established. The deterioration of odor can be determined according to the screened evaluation metabolic marker. The method is of a great research value, and provides a new idea for controlling lipid oxidation and reducing the generation of bad odor.

The embodiments of the present disclosure will be described in detail below to make these and other aspects of the present disclosure more clearly understood. It should be understood that the above general description and the detailed description below are merely illustrative and explanatory, and are not intended to limit the present disclosure.

### BRIEF DESCRIPTION OF THE DRAWINGS

In order to more clearly illustrate the embodiments of the present disclosure or the technical solutions in the prior art, the accompanying drawings required for use in the embodiments or the description of the prior art will be briefly introduced below. Obviously, the accompanying drawings merely illustrate some of the embodiments of the present disclosure. For those of ordinary skill in the art, other embodiments can be obtained based on the drawings of the disclosure without making creative efforts.

FIG. 1 is a flow chart of a method for determining odor deterioration and lipid oxidation degree of tilapia during cold storage in accordance with an embodiment of the present disclosure;

FIG. 2 is a flow chart of step (S20) in the method in accordance with an embodiment of the present disclosure; and

FIG. 3 illustrates a lipid metabolic network and related metabolic pathways in accordance with an embodiment of the present disclosure.

### DETAILED DESCRIPTION OF EMBODIMENTS

The technical solutions in the embodiments of the present disclosure will be clearly and completely described below with reference to the accompanying drawings of the present disclosure. Obviously, the described embodiments are some embodiments of the present disclosure, rather than all embodiments. Based on the embodiments described herein, all other embodiments obtained by those skilled in the art without making creative efforts shall fall within the scope of the present disclosure.

The flowcharts shown in the accompanying drawings are merely illustrative, and do not necessarily include all the contents and operations/steps, nor must they be executed in the order described. For example, some operations/steps may also be decomposed, combined or partially merged, and thus the actual execution order may change according to actual conditions.

It should be understood that terms used herein are only intended to describe the embodiments, and are not intended to limit the present disclosure. As used herein, the singular forms “a”, “an” and “the” are intended to include plural forms unless otherwise defined.

Specifically, the embodiments of the present disclosure will be further described below in conjunction with the accompanying drawings.

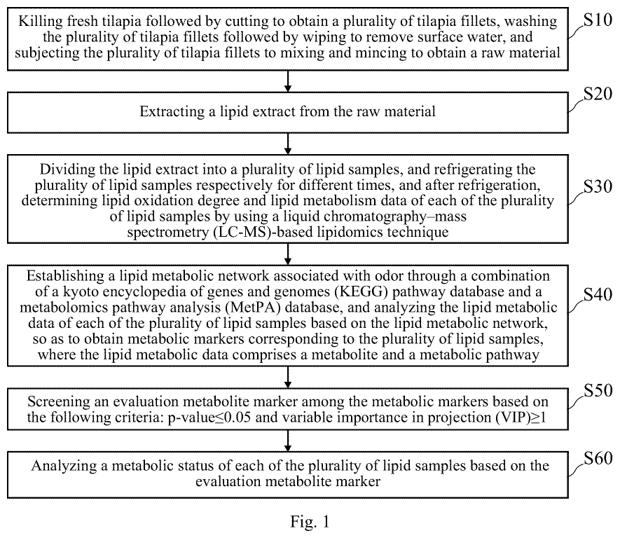

FIG. 1 is a flow chart of a method for determining odor deterioration and lipid oxidation degree of tilapia during cold storage. As shown in FIG. 1, the method includes steps (S10)-(S60).

(S10) Fresh tilapia are killed and cut to obtain a plurality of tilapia fillets. The plurality of tilapia fillets are washed, wiped to remove surface water, mixed and minced to obtain a raw material.

In an embodiment, the step (S10) is performed as follows.

The fresh tilapia is purchased from Luchaogang Aquatic Products Wholesale Market in Pudong New Area, Shanghai, which have shiny scales, clear gill filaments, bright red gills, clear and transparent mucus on body surfaces and inside gills, no odor and roughly consistent size and weight. The fresh tilapia is placed in a foam box with crushed ice and oxygen for transportation. The fresh tilapia is stunned by physical knocking, killed, and subjected to removal of head and internal organs and cutting to obtain the plurality of tilapia fillets. The plurality of tilapia fillets are washed, wiped to remove surface moisture, mixed and minced to obtain the raw material for subsequent experiments.

(S20) A lipid extract is extracted from the raw material.

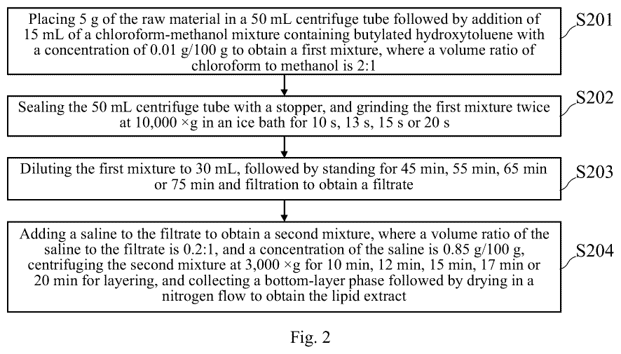

Referring to FIG. 2, in an embodiment, the step (S20) is performed as follows.

(S201) 5 g of the raw material is placed in a 50 mL centrifuge tube, and 15 mL of a chloroform-methanol mixture containing butylated hydroxytoluene with a concentration of 0.01 g/100 g is added to obtain a first mixture, where a volume ratio of chloroform to methanol is 2:1.

(S202) The centrifuge tube is sealed with a stopper, and the first mixture is ground twice at 10,000×g in an ice bath for 10 s, 13 s, 15 s or 20 s.

(S203) The first mixture is diluted to 30 mL, stood for 45 min, 55 min, 65 min or 75 min and filtration to obtain a filtrate.

(S204) The filtrate is added with a saline to obtain a second mixture, where a volume ratio of the saline to the filtrate is 0.2:1, and a concentration of the saline is 0.85 g/100 g. The second mixture is centrifugated at 3,000×g for 10 min, 12 min, 15 min, 17 min or 20 min for layering. A bottom-layer phase is collected and dried in a nitrogen flow to obtain the lipid extract.

(S30) The lipid extract is divided into a plurality of lipid samples. The plurality of lipid samples are refrigerated for different times, respectively. After the refrigeration is completed, lipid oxidation degree and lipid metabolism data of each of the plurality of lipid samples are determined by using a liquid chromatography-mass spectrometry (LC-MS)-based lipidomics technique.

In some embodiments, the plurality of lipid samples are each independently refrigerated at 0° C., 2° C., 4° C., 6° C. or 8° C.

In some embodiments, in step (S30), the lipid extract is divided into four lipid samples, and the four lipid samples are refrigerated at 4° C. for 0 days, 3 days, 9 days and 15 days, respectively.

In some embodiments, the LC-MS-based lipidomics technique is performed as follows.

0.5 mL of a lipid sample is added to a 2 mL centrifuge tube followed by addition of 600 μL of a methanol solution containing 4 ppm of 4-chloro-L-phenylalanine maintained at −20° C. and vortex mixing to obtain a mixture. The vortex mixing is performed for 25 s, 28 s, 30 s, 33 s or 35 s.

The mixture is homogenized by a tissue homogenizer containing 100 mg of glass beads at 60 Hz for 80 s, 85 s, 90 s, 95 s or 100 s.

The mixture is centrifuged at 12,000×g and 4° C. for 10 min followed by ultrasonic treatment at room temperature for 7 min, 8 min, 10 min, 12 min or 13 min. A supernatant is collected, and then filtered with a 0.22-μm membrane filter to obtain a filtrate.

The filtrate is transferred to a vial followed by LC-MS analysis and chromatography. A liquid chromatography analysis is performed using an Ultimate 3000 UHPLC system (Thermo Fisher Scientific, USA) to obtain total ion maps and metabolic data. Calculation is performed according to various analytical methods. Chromatography is performed using an ACQUITY UPLC® HSST3 (150×2.1 mm, 1.8 μm) (Waters, Milford, MA, USA). The column is maintained at 40° C. An injection volume is calibrated to 2 μL, and an injection flow rate is calibrated to 0.25 mL/min. Acetonitrile with 0.1% formic acid (v/v) (C) and water with 0.1% formic acid (v/v) (D) are used as mobile phases for a LC-electrospray ionization (ESI) (+)-MS analysis. Acetonitrile (A) and an ammonium formate solution (5 mM) (B) are used as analytes for the LC-ESI (−)-MS analysis. The metabolite detection is performed by mass spectrometry using an ESI ion source and QExactive (ThermoFisherScientific, USA). Data is acquired using data-dependent MS/MS and simultaneous MS1 and MS/MS (full MS-ddMS2 mode). Parameters are set as follows. a capillary temperature is 325 C.°, a MS1 range is m/z 100-1000, a MS1 resolution is 70,000 FWHM, the number of data-dependent scans per cycle is 10, an MS/MS resolving power is 17,500 FWHM, and a normalized collision energy is 30%.

(S40) A lipid metabolic network associated with odor is established through a combination of a kyoto encyclopedia of genes and genomes (KEGG) pathway database and a metabolomics pathway analysis (MetPA) database. The lipid metabolic data of each of the plurality of lipid samples is analyzed based on the lipid metabolic network, so as to obtain metabolic markers corresponding to the plurality of lipid samples. The lipid metabolic data includes a metabolite and a metabolic pathway.

It should be noted that the KEGG database integrates genomic, chemical and system functional information, which contains a large amount of useful information. The genomic information is stored in a GENES database, including complete and partially sequenced genomic sequences. More advanced functional information is stored in a PATHWAY database, including diagrams of cell biochemical processes such as metabolism, membrane transport, signal transduction, cell cycle, and homologous conserved sub-pathways. Another KEGG database is a LIGAND database, which includes information about chemical substances, enzyme molecules, enzyme reactions, etc. The integrated metabolic pathway query provided by KEGG includes the metabolism of carbohydrates, nucleosides, amino acids, etc. and the biodegradation of organic matter. This not only provides all possible metabolic pathways, but also comprehensively annotates enzymes involved in catalyzing each step of the reaction, including amino acid sequences, links to protein data bank (PDB) libraries, etc. KEGG is a powerful tool for in vivo metabolic analysis and metabolic network research, and facilitates the investigation of genes and expression information thereof as a whole network. MetPA is part of metaboanalyst, and is mainly predicated on the KEGG metabolic pathway. The MetPA database can identify possible metabolic pathways affected by biological disturbances through metabolic pathway enrichment and topological analysis, thereby analyzing the metabolic pathway of the metabolite. The MetPA database can be adopted to analyze a related metabolic pathway of a differential metabolite. A hypergeometric test is used as a data analysis algorithm, and a Relative-betweeness Centrality is used as a metabolic pathway topology structure. The lipid metabolic network associated with odor is established through the combination of the KEGG metabolic pathway and the MetPA database. The above metabolic markers are entered into an MetPA dialogue box. A compound name is selected in an Input label, and Submit is clicked. A species, a hypergeometric test and a pathway topology analysis are selected. A relative-betweenness centrality is adopted, and a registration is performed for pathway model analysis. The KEGG database is configured for signal pathway analysis, and an Interactive Pathways Explorer is configured to visualize a metabolite pathway. The differences between the method and other analyses are as follows. Firstly, there is a difference in the analysis objects. In this application, the lipid extract is extracted separately for metabolic analysis, which is the first case in this field. The interference of other factors is eliminated, resulting in a more reliable analysis result. Secondly, the analysis adopts different metabolic databases. Conventional databases are predicated on the KEGG database for analysis. For the method of the present disclosure, the MetPA database is adopted for topological analysis based on the KEGG database to identify metabolic pathways related to the investigation, the data and the metabolic model are analyzed through hypergeometric test and the relative-betweenness centrality, and the metabolic network is visualized through the Interactive Pathways Explorer online analysis tool.

(S50) An evaluation metabolite marker is screened among the obtained metabolite markers based on the following criteria: p-value ≤0.05 and variable importance in projection (VIP)≥1. The p-value refers to the probability of a more extreme result than the obtained sample observation when the null hypothesis is true, which is a parameter used to determine the result of the hypothesis test, and is a significance level calculated based on an actual statistic. The VIP value is an indicator to measure the importance of a variable to a model. It describes an overall contribution of each variable to the model, and is normally configured to measure the influence and explanatory power of the expression pattern of each metabolite on the classification and discrimination of each group of samples, so as to explore differential metabolites with biological significance.

It should be noted that variables with VIP values greater than 1 are used as candidate variables for biomarkers. In order to verify whether the candidate variables found in multidimensional statistics had significant differences in unit statistics, a T test is adopted in the experiment, where P<0.05 indicates a significant difference. The candidate variables are selected in combination with a loading diagram, a search, match and speculation in databases such as METLIN, KEGG, PubChem, etc., are performed according to mass spectrometry information of compounds represented by these variables, so as to determine possible biomarkers. The evaluation metabolite marker is screened according to the above conditions, which is 1-hexadecanol, 1-monopalmitate, 10-heptadecenoic acid, 13-docosenamide, 5,8,11-eicosatrienoic acid, 7,10,13,16,19-docosapentaenoic acid, 9-octadecenoic acid, 9,12-octadecadienoic acid, arachidonic acid, ethyl 4-ethoxybenzoate, campesterol, dibutyl phthalate, glycerol monostearate, heptadecanoic acid, myristic acid, oleic acid, palmitelaidic acid, palmitic acid or a combination thereof.

(S60) A metabolic status of each of the plurality of lipid samples is analyzed based on the evaluation metabolite marker.

In this embodiment, the step (S60) is performed as follows.

Three samples are randomly selected among the plurality of lipid samples as parallel groups on day 0, day 3, day 9 and day 15, respectively, so as to analyze lipid oxidation degree and lipid metabolism.

The lipid extracted from tilapia is stored in a refrigerated environment at 4 C.° for different days. The lipid metabolic network associated with the odor of tilapia is established through analysis using the LC-MS-based lipidomics technique, and 18 metabolic markers are screened, including 1-hexadecanol, 1-monopalmitate, 10-heptadecenoic acid, 13-docosenamide, 5,8,11-eicosatrienoic acid, 7,10,13,16,19-docosapentaenoic acid, 9-octadecenoic acid, 9,12-octadecadienoic acid, arachidonic acid, ethyl 4-ethoxybenzoate, campesterol, dibutyl phthalate, glycerol monostearate, heptadecanoic acid, myristic acid, oleic acid, palmitelaidic acid and palmitic acid. The degree of lipid oxidation and deterioration of flavor quality can be judged by the content of these markers.

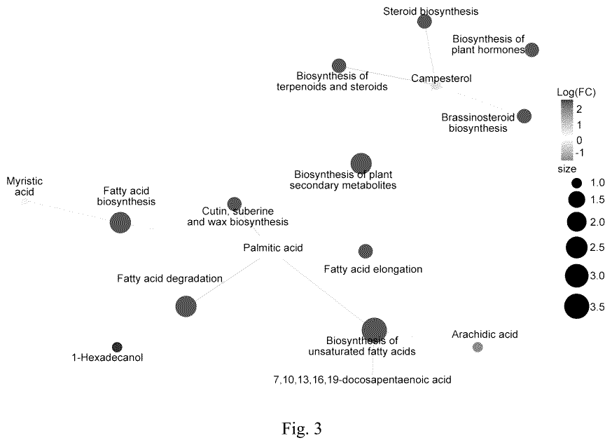

The metabolite markers identified by lipid metabolomics analysis are shown in Table 1. In the present disclosure, the lipid metabolic network diagram associated with odor is constructed (as shown in FIG. 3), the association between lipid metabolism and odor deterioration is established, and the odor deterioration is determined by screening of metabolic markers. The method of the present disclosure is of great research value, and provides a new idea for controlling lipid oxidation and reducing the generation of bad odors.

TABLE 1
Metabolic markers identified through lipidomics technique

| | Ionic strength | Ionic strength | | | Metabolic | |
|---|---|---|---|---|---|---|
| Name | on day 0 | on day 15 | P-value | VIP | pathway | Difference |
| 1-Hexadecanol | 402759.16 | 64116.79 | 0.00071 | 1.36 | C00823 | Up |
| 1-Palmitate | 15295379.9 | 18625951 | 0.00290 | 1.30 | | Down |
| 10-Heptadecenoic | 2752042.23 | 3524326.61 | 0.01362 | 1.23 | | Down |
| acid | | | | | | |
| 13-Behenamide | 934608.74 | 1581085.08 | 0.01462 | 1.22 | | Down |
| 5,8,11- | 13294038.1 | 12091630 | 0.00333 | 1.29 | | Up |
| Docosatrienoic | | | | | | |
| acid | | | | | | |
| 7,10,13,16,19- | 16960825.9 | 16289194.6 | 0.00545 | 1.28 | C16513 | Up |
| Docosapentaenoic | | | | | | |
| acid | | | | | | |
| 9-Octadecenoic | 123657427 | 110792055 | 0.01323 | 1.23 | | Up |
| acid | | | | | | |
| 9,12- | 101713290 | 90024942.8 | 0.00129 | 1.32 | | Up |
| Octadecadienoic | | | | | | |
| acid | | | | | | |
| Arachidonic acid | 530563.68 | 1541643.66 | 0.00152 | 1.31 | C06425 | Down |
| Ethyl 4- | 733276.87 | 659082.01 | 0.01134 | 1.24 | | Up |
| ethoxybenzoate | | | | | | |
| Campesterol | 1054911.19 | 1483203.54 | 0.01247 | 1.24 | C01789 | Down |
| Dibutyl phthalate | 1280451.24 | 64116.79 | 0.00007 | 1.36 | C14214 | Up |
| Glyceryl | 2487390.32 | 3751638.59 | 0.00002 | 1.35 | | Down |
| monostearate | | | | | | |
| Heptadecanoic | 925000.26 | 1388345.35 | 0.00489 | 1.28 | | Down |
| acid | | | | | | |
| Myristic acid | 12939554.8 | 11136389.3 | 0.02106 | 1.19 | C06424 | Up |
| Oleic acid | 64116.79 | 31098260 | 0.00008 | 1.36 | | Down |
| Palmitelaidic aicd | 44091224 | 38265434.6 | 0.01817 | 1.20 | | Up |
| Palmitic acid | 103597801 | 95243514 | 0.00989 | 1.25 | C00249 | Up |

It should be understood that the above steps are described in a certain order, but are not necessarily performed in sequence in the above order. Unless there is clear explanation in this article, the execution of these steps does not have strict order restriction, and these steps can be performed in other orders. Moreover, some of the steps in the embodiments may include multiple steps or multiple stages, which are not necessarily performed at the same time, and can be executed at different times. These steps or stages is not necessarily executed in sequence, and can be executed in turn or alternately with other steps or at least part of the steps or stages in other steps.

It should be understood that the singular form “a” or “an” used herein is intended to include the plural form as well, unless an exception is clearly supported. It should also be understood that the term “and/or” used herein refers to any and all possible combinations of one or more items listed in association. The serial numbers of the above embodiments are merely for description, and do not represent the advantages and disadvantages of the embodiments.

Those of ordinary skill in the art should understand that the embodiments described above are merely illustrative of the present disclosure, and are not intended to imply that the scope of the present disclosure (including the claims) is limited to these embodiments. Within the idea of the present disclosure, the above embodiments or technical features therein can also be combined. Therefore, any modifications, equivalent substitutions and improvements made without departing from the spirit of the disclosure shall fall within the scope of the disclosure defined by the appended claims.

What is claimed is:

1. A method for determining odor deterioration and lipid oxidation degree of tilapia during cold storage, comprising:
step (1) killing fresh tilapia followed by cutting to obtain a plurality of tilapia fillets, washing the plurality of tilapia fillets followed by wiping to remove surface water, and subjecting the plurality of tilapia fillets to mixing and mincing to obtain a raw material;
step (2) extracting a lipid extract from the raw material;
step (3) dividing the lipid extract into a plurality of lipid samples, and refrigerating the plurality of lipid samples respectively for different times; and after refrigeration, determining lipid oxidation degree and lipid metabolism data of each of the plurality of lipid samples by using a liquid chromatography-mass spectrometry (LC-MS)-based lipidomics technique;
step (4) establishing a lipid metabolic network associated with odor through a combination of a kyoto encyclopedia of genes and genomes (KEGG) pathway database and a metabolomics pathway analysis (MetPA) database, and analyzing the lipid metabolic data of each of the plurality of lipid samples based on the lipid metabolic network, so as to obtain metabolic markers corresponding to the plurality of lipid samples; wherein the lipid metabolic data comprises a metabolite and a metabolic pathway;
step (5) screening an evaluation metabolite marker among the metabolic markers based on the following criteria: p-value ≤0.05 and variable importance in projection (VIP)≥1; and
step (6) analyzing a metabolic status of each of the plurality of lipid samples based on the evaluation metabolite marker.

2. The method of claim 1, wherein the step (1) is performed through a step of:
placing the fresh tilapia in a foam box with crushed ice and oxygen for transportation, stunning the fresh tilapia by physical knocking, killing the fresh tilapia followed by removal of head and internal organs and cutting to obtain the plurality of tilapia fillets, washing the plurality of tilapia fillets followed by wiping to remove surface water; and mixing and mincing the plurality of tilapia fillets to obtain the raw material.

3. The method of claim 1, wherein the step (2) is performed through steps of:
step (2.1) placing 5 g of the raw material in a 50 mL centrifuge tube followed by addition of 15 mL of a chloroform-methanol mixture containing 0.01 wt. % of butylated hydroxytoluene to obtain a first mixture; wherein a volume ratio of chloroform to methanol is 2:1;
step (2.2) sealing the 50 mL centrifuge tube with a stopper, and grinding the first mixture twice at 10,000×g in an ice bath for 10 s, 13 s, 15 s or 20 s;
step (2.3) diluting the first mixture to 30 mL, followed by standing for 45 min, 55 min, 65 min or 75 min and filtration to obtain a filtrate; and
step (2.4) adding a saline to the filtrate to obtain a second mixture, wherein a volume ratio of the saline to the filtrate is 0.2:1, and a concentration of the saline is 0.85 g/100 g; centrifuging the second mixture at 3,000×g for 10 min, 12 min, 15 min, 17 min or 20 min for layering; and collecting a bottom-layer phase followed by drying in a nitrogen flow to obtain the lipid extract.

4. The method of claim 1, wherein in step (3), the lipid extract is divided into four lipid samples, and the four lipid samples are refrigerated at 4° C. for 0 day, 3 days, 9 days and 15 days, respectively.

5. The method of claim 1, wherein in step (3), the LC-MS-based lipidomics technique is performed through steps of:
adding 0.5 mL of each of the plurality of lipid samples to a 2 mL centrifuge tube followed by addition of 600 μL of a methanol solution containing 4 ppm of 4-chloro-L-phenylalanine maintained at −20° C. and vortex mixing for 30 s to obtain a mixture;
homogenizing, by a tissue homogenizer containing 100 mg of glass beads, the mixture at 60 Hz for 90 s;
centrifuging the mixture at 12,000×g and 4° C. for 10 min followed by ultrasonic treatment at room temperature for 7 min, 8 min, 10 min, 12 min or 13 min, and collecting a supernatant; and filtering the supernatant with a 0.22-μm membrane filter to obtain a filtrate; and
transferring the filtrate to a vial followed by LC-MS analysis.

6. The method of claim 1, wherein the step (6) is performed through a step of:
randomly selecting three lipid samples among the plurality of lipid samples respectively on day 0, day 3, day 9 and day 15 as parallel groups to analyze lipid oxidation degree and lipid metabolism.

7. The method of claim 1, wherein the evaluation metabolic marker is selected from the group consisting of 1-hexadecanol, 1-monopalmitate, 10-heptadecenoic acid, 13-docosenamide, 5,8,11-eicosatrienoic acid, 7,10,13,16,19-docosapentaenoic acid, 9-octadecenoic acid, 9,12-octadecadienoic acid, arachidonic acid, ethyl 4-ethoxybenzoate, campesterol, dibutyl phthalate, glycerol monostearate, heptadecanoic acid, myristic acid, oleic acid, palmitelaidic acid, palmitic acid and a combination thereof.
